(19) United States
(12) Patent Application Publication
(10) Pub. No.: US 2023/0248871 A1
(43) Pub. Date: Aug. 10, 2023

(54) IOT BASED VERTICAL AUTOCLAVE MACHINE

(71) Applicants: Surya Kalyan Chakravarthy Nidamanuri, Ongole (IN); Jafar Ali Ibrahim Syed Masood, Vellore (IN); Sasmita Samanta, Bhubaneswar, Odisha (IN)

(72) Inventors: Jafar Ali Ibrahim Syed Masood, Vellore (IN); Surya Kalyan Chakravarthy Nidamanuri, Ongole (IN); Chee Kuang Kok, Melaka (MY); Chinnaiyan Ramasubramanian, Bengaluru (IN); Hanumantha Rao Yarrapathruni Venkata, Ongole (IN); Navaneethakrishnan Gopal, Ongole (IN); Dhivagar Ramasamy, Ongole (IN); Venkat Rao Chundru, Ongole (IN); Anka Babu Kamma, Ongole (IN); Satish Kumar Harasurakar, Ongole (IN); Abhishek Kumar Naresh Prasad Malakar, Ongole (IN); Kalyana Krishna Yerru, Ongole (IN); Srihari Mallela, Ongole (IN); Manikanta Narala, Ongole (IN); Ganesh Lingala, Ongole (IN)

(21) Appl. No.: 18/301,645

(22) Filed: Apr. 17, 2023

(51) Int. Cl. A61L 2/24; A61L 2/04; G16Y 40/10; G16Y 10/60; G16Y 20/10

(52) U.S. Cl. CPC A61L 2/24; A61L 2/04; G16Y 10/60; G16Y 20/10; G16Y 40/10

(57) ABSTRACT

The present invention relates to a system (100) for IOT based vertical autoclave machine. The system (100) comprises a plurality of sensors, a control unit, a memory, a digital temperature indicator, a water level indicator, a pressure indicator a digital timer, a cloud and a user interface. The plurality of sensors used to measure the sterilization parameters of autoclave machine. The control unit allows to set the sterilization temperature, pressure and water level of autoclave machine. The digital temperature indicator used to display the temperature of machine accurately. The water level indicator displays the volume of water inside the chamber of machine. The pressure indicator shows the pressure inside the machine for sterilization. The power supply used to provide supply to the system. The cloud enables storing data and files on the internet of autoclave machine. The user interface allows interaction of user with application or with website.

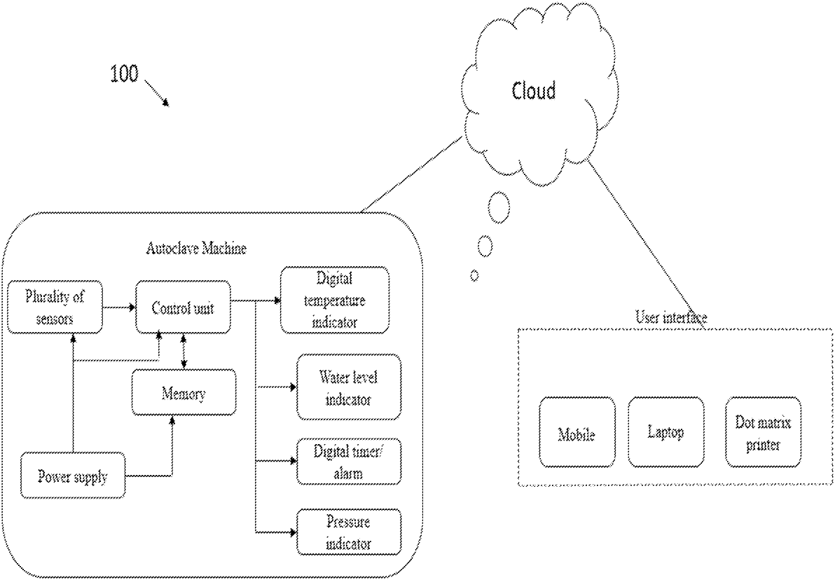

### FIELD

The present invention relates to a field of internet of things and particularly a system to monitor vertical autoclave machine based on IOT.

### BACKGROUND

An autoclave is a device used in industrial and scientific procedures that demand for higher temperature and pressure in comparison to ambient temperature. In addition to being used in the chemical industry to vulcanize rubber and cure coatings, autoclaves are also utilized in the medical field to sterilize instruments prior to surgery. In the manufacture of composite materials, industrial autoclaves are utilized often. Therefore, system is required for the successful implementation of reliable steam sterilization and monitors or manages the autoclave machines for future use.

### SUMMARY

The following presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

The present invention is generally directed to IOT based vertical autoclave machine. The aim of this system is to meet effective sterilization requirements in bacteriological and research laboratories, hospitals and clinics and food and beverage units. The system maximizes space utilization and uses microprocessor control system which enables comfortable working all the time while operating the machine. The system is fabricated using GMP stainless steel double and triple walled construction; in addition, manufacturing of vertical autoclaves with other configurations, which include stainless steel inner and exterior made of mild teel (MS).

Some of the objects of the present disclosure, which at least one embodiment herein satisfies, are as follows.

It is an object of the present disclosure to ameliorate one or more problems of the prior art or to at least provide a useful alternative

An object of the present disclosure is to provide a system that enable guaranty of safe and efficient sterilization even in confined spaces.

An object of the present disclosure is to provide a system which meets all relevant guidelines and standards such as NABH standards for sterilization.

Another object of the present disclosure is to provide a system that offers IQ / OQ / PQ calibration documentation with extra cost.

Still another object of the present disclosure is to provide a system that is fully customizable and provided with large scope of customization to meet individual budget.

Another object of the present disclosure is to provide a system that utilizes optional accessories such as temperature chart recorder, digital temperature indicator, PID controller and provision to attach a dot matrix printer.

Other objects and advantages of the present disclosure will be more apparent from the following description, which is not intended to limit the scope of the present disclosure.

### BRIEF DESCRIPTION OF THE DRAWINGS

The FIGURE illustrates the block diagram of IOT based vertical autoclave machine, in accordance with an embodiment of the present invention.

### DETAILED DESCRIPTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention.

The FIGURE illustrates the block diagram of IOT based vertical autoclave machine, in accordance with an embodiment of the present invention. The system (100) comprises a plurality of sensors, a control unit, a memory, a digital temperature indicator, a water level indicator, a pressure indicator a digital timer, a cloud and a user interface. The plurality of sensors used to measure the sterilization parameters of autoclave machine. The control unit allows to set the sterilization temperature, pressure and water level of autoclave machine. The digital temperature indicator used to display the temperature of machine accurately. The water level indicator displays the volume of water inside the chamber of machine. The pressure indicator shows the pressure inside the machine for sterilization. The power supply used to provide supply to the system. The cloud enables storing data and files on the internet of autoclave machine. The user interface allows interaction of user with application or with website.

Standard models of vertical autoclave designed to available with chamber volumes of 22 liters to 175 liters that accommodate two and three drums accordingly. These systems are designed for meeting effective sterilization requirements in bacteriological and research laboratories, hospitals and clinics and food and beverage units. The vertical chamber design maximizes space utilization, while microprocessor control system enables comfortable working all the time you operate the machine.

Vertical type autoclaves deliver flexible solutions of steam sterilization in hospitals, clinics and other areas of application, where reliable steam sterilization is a mandatory process. Vertically designed chamber provides guaranty of safe and efficient sterilization even in confined spaces. In order to suit specific application, these units are fabricated as fully GMP stainless steel double and triple walled construction; in addition, we also manufacture vertical autoclaves with other configurations, which include stainless steel inner and exterior made of Mild Steel (MS).

The most important feature that keeps our range of vertical autoclaves top of others is meeting all relevant guidelines and standards such as NABH standards. As an option, we also provide IQ / OQ / PQ calibration documentation with extra cost. These systems are fully customizable and provided with large scope of customization; to meet individual budget, we can equipment your vertical autoclave with a number of optional accessories such as temperature chart recorder, digital temperature indicator, PID Controller and provision to attach a dot matrix printer.

Features:
Tank volume 20 to 175 liters
Equipped with joint less silicon rubber gasket provided with immersion type heater
Double, triple wall & GMP construction, comply with NABL standards, Radial or wing nut type door locking, supply with drums (extra cost), water level indicator, easy operation and low maintenance, digital temperature indicator, IQ / OQ / PQ Documentation
Temperature chart recorder, provision to attach dot matrix printer, USB interface for data transfer.

Specifications:
MODEL BT/VA-22 BT/VA-40 BT/VA-50 BT/VA-98 BT/VA-175
Internal Tank Capacity 22 Liter 40 Liter 50 Liter98 Liter 175 Liter
Load 2.00 KW 3.00 KW 3.00 KW 4.00 KW 6.00 KW
Chamber dimension 10″ x 18″ 12″ x 22″ 14″ x 22″ 18″ x 24″ 22″ x 36″
No. of Drums & Sizes -2 (9″ x11″) 2 (9″ x11″) 2 (12″ x 15″) 3 (12″ x 15″)
Working Pressure 1.2 kgf /cm sq. g (15 psi - 17 psi) (Can be Upgraded up to 30 PSI)
Sterilizing Temperature 121° C. to 134° C. (150° C. - Optional)
Heat Average <=±1° C.
Sterilization Time 25 to 30 Minutes
Digital Timer 0 to 99 min
Construction Double / Triple wall & SS GMP construction
Outer Chamber Mild Steel (304 stainless Steel - Optional)
Inner Chamber 304 SS (316 / 316 L grades Stainless Steel - Optional)
Insulation Glass Wool
Waste Container Stainless Steel
Door / Lid Foot lifted with safety and interlock device
Door Locking type Radial Type (Wing Nut Optional)
Water Filling & Removal Manual
Safety Features Radial locking, safety valve, low water detector and pressure interlock
Optional accessories
   PID controller
   Low / High water cut-off device
   Digital alarm
   Temperature chart recorder
   Dot Matrix Printer
   RS 232 / USB PC Connection Port
   NABL certification
   Stainless Steel wire mesh carrier
   Dressing drum
   Sterilization indicator tape
   Autoclave bags
   PLC control system
   IQ / OQ / PQ Documentation
-On Side grantee charge extra
-Power Supply 220 Volts / 440 Volts

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

What is claimed is:

1. A system (100) for internet of things based vertical autoclave machine comprising:
a plurality of sensors used to measure the sterilization parameters of autoclave machine, comprises:
a temperature sensor used to maintain stable temperature of autoclave machine;
a pressure sensor used to monitor the steam pressure inside the autoclave machine;
a water level sensor is used to monitor the water level in autoclave machine;
a control unit allows to set the sterilization temperature, pressure and water level of autoclave machine;
a memory stores sensors data information for performing various operations;
a digital temperature indicator used to display the temperature of machine accurately;
a water level indicator displays the volume of water inside the chamber of machine; a pressure indicator shows the pressure inside the machine for sterilization;
a digital timer activates alarm based on different operating conditions of autoclave machine;
a power supply used to provide supply to the system;
a cloud enables storing data and files on the internet of autoclave machine;
a user interface allows interaction of user with application or with website.

2. The system (100) of claim 1, wherein the water level indicator displays the water level inside a chamber with capacities ranging from 22 to 175 liters, which may accommodate two or three drums, respectively.

3. The system (100) of claim 1, wherein the machine is fabricated by fully GMP stainless steel double and triple walled construction and also with stainless steel inner and exterior made of mild steel (MS).

4. The system (100) of claim 1, wherein the user interface includes mobile, laptop and dot matrix printer. The dot matrix printer shows temperature chart recorder data.

5. The system (100) of claim 1, wherein the IOT based vertical autoclave machine has been explored for meeting effective sterilization requirements in bacteriological and research laboratories, hospitals and clinics and food and beverage units.
